(19) United States
(12) Patent Application Publication
(10) Pub. No.: US 2002/0049469 A1
(43) Pub. Date: Apr. 25, 2002

(54) Acupressure foot board

(76) Inventor: Jey Won Chang, Seoul (KR)

Correspondence Address: EDWARD A SOKOLSKI, 3868 CARSON STREET, 105, TORRANCE, CA, 90503, US

(21) Appl. No.: 10/022,656

(22) Filed: Dec. 14, 2001

Related U.S. Application Data
(60) Provisional application No. 60/233,905, filed on Sep. 20, 2000.

(30) Foreign Application Priority Data
Dec. 15, 2000 (KR) 0-00351003

(51) Int. Cl. A61B 17/00

(52) U.S. Cl. 606/204

(57) ABSTRACT

A foot board has a plurality of pressure rods extending upwardly therefrom which are positioned within outlines of average foot sizes for men and women. The pressure rods are positioned at reflex points for attaining acupressure treatment when the user stands on the rods with his or her feet oriented within the appropriate foot outlines. The user can change the rods to provide the needed intensity to make for greater pressure on the needed reflex points and more intense massaging. Also, thermal pressure rods or vibrating pressure rods activated electrically can be installed.

### BACKGROUND OF THE INVENTION

1. Field of the Invention

This invention relates to acupressure devices and more particularly to a foot board having pressure rods extending upwardly therefrom on which the user can stand with the rods abutting against predetermined reflex points on the soles of the user's feet.

2. Description of the Related Art

Many instruments are available for massaging reflex points on foot soles. Such instruments are described, for example in U.S. Pat. No. 5,860,229 issued Jan. 19, 1999 to Morgenstern; U.S. Pat. No. 5,158,073 issued Oct. 27, 1992 to Bukowski; U.S. Pat. No. 5,263,474 issued Nov. 23, 1993 to Agader; and U.S. Pat. No. 6,261,306 issued Jul. 17, 2001 to Kramer. These devices generally have uniform rods which are not arranged to provide proper effects in that they are often randomly oriented and not oriented at the correct reflex points. Further, they generally do not provide for changing of the rods to accommodate different requirements of the user. In addition, prior art devices generally utilize uniform rods for the entire set of rods which does not accommodate for individual requirements of the user. It is well established that as the reflex points are very sensitive, if the wrong reflex points are massaged or are massaged with incorrect intensity the needed effects cannot be obtained.

### SUMMARY OF THE INVENTION

The device of the present invention is formed from a base board which has two sets of left and right foot outlines formed thereon. One of these sets of foot outlines is larger than the other and can generally be used for men while the other is smaller so as to accommodate women and children or men with smaller feet. Holes are formed within the foot outlines corresponding to the locations of various known pressure points on the soles of the feet. The holes have different depths so as to accommodate different sizes and types of pressure rods in different predetermined holes. The rods may be of plastic, wood, jade, ocher, or metallic magnetic material.

The heights of the normal pressure rods can be increased by installing pressure caps thereover. These caps can be installed as may be desired over any of the regular rods to provide greater pressure as may be required by the particular user. Different diameter rods can also be utilized as well as thermal pressure rods or vibrating pressure rods to accommodate the particular requirements of each user.

The rods are located within the foot pattern at reflex points, the rods being chosen as to size and characteristic and installed to provide optimum pressure for each such reflex point. Thus, various pressure intensities can be provided by selecting the rods as to height and thickness. Also, thermal and vibrating equipment can be connected to the pressure rods to increase their efficacy.

It is therefore an object of this invention to provide an improved device for massaging the soles of one's feet at the acupressure reflex points.

It is a further object of this invention to provide a foot massaging device in which the pressure provided at each reflex point can be adjusted for optimum results.

Other objects of the invention will become apparent as the description proceeds in connection with the accompanying drawings.

### DESCRIPTION OF THE DRAWINGS

FIG. 1 is a top front perspective view of a preferred embodiment of the invention;

FIG. 2 is a top front perspective view of the preferred baseboard;

FIG. 2A is a side elevational view showing various pressure rods which may be employed;

FIG. 3A is a cross sectional view illustrating a typical set of pressure rods installed in the base board; and

FIG. 3B is a view similar to FIG. 3A but including a thermal and vibration adapter.

### DETAILED DESCRIPTION OF THE INVENTION

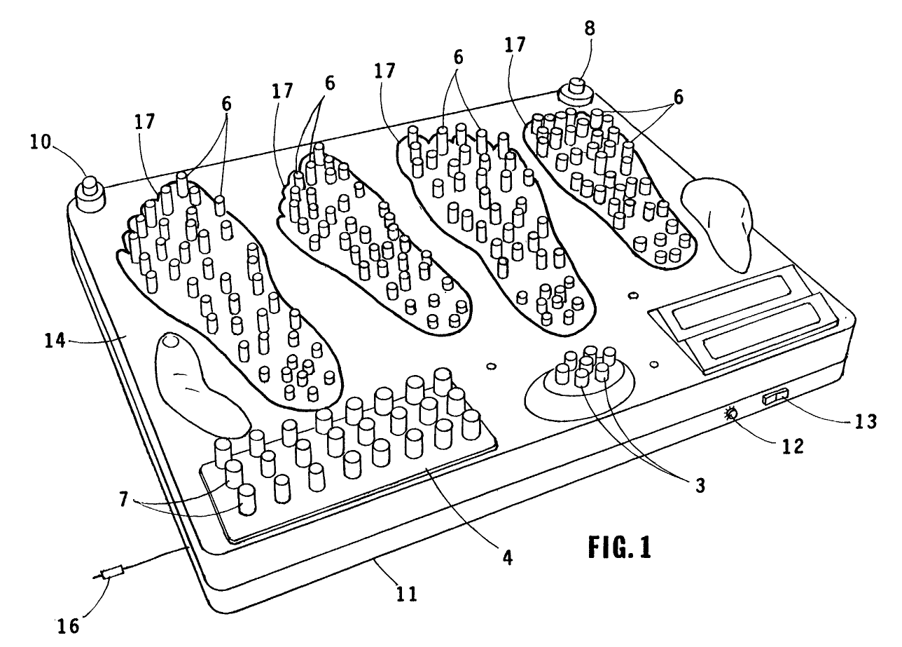

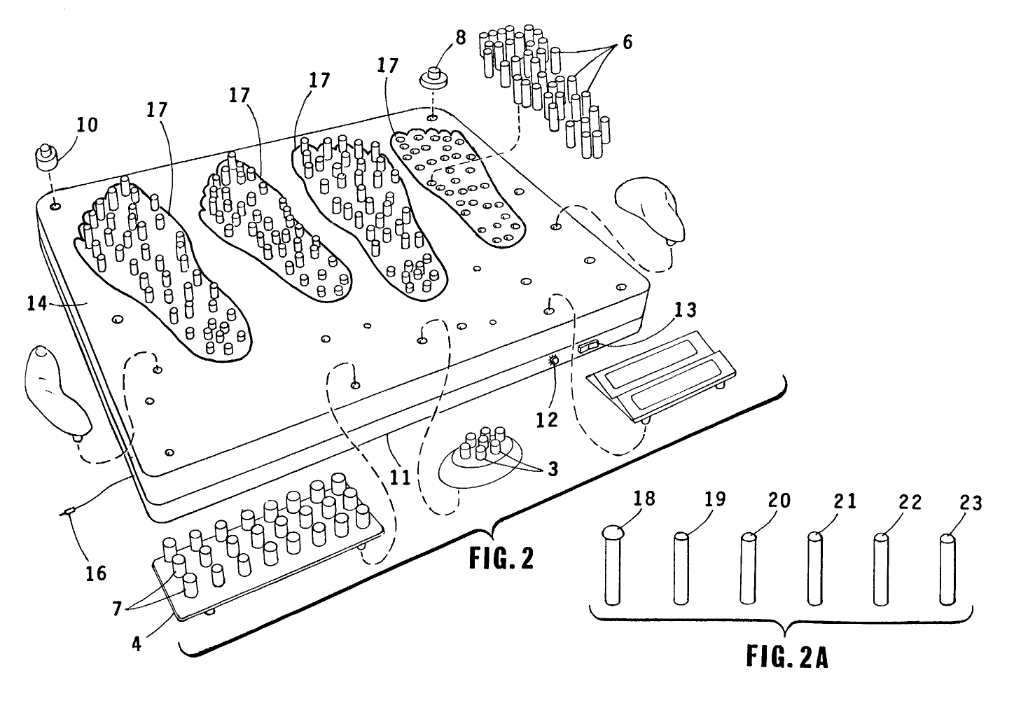

Referring now to FIGS. 1 and 2, the device of the invention is fabricated by attaching foot shaped films 17 about 0.5 mm thick to the base board. Two of these films are of a size to match typical female feet while the other two match typical male feet. Holes are formed in he board and film, each located in a position corresponding to a reflex point on the soles of the feet. Pressure rods are installed in the holes, the holes having a depth which corresponds to the particular pressure required for each pressure point. In the preferred embodiment, 41 holes are formed in the male foot patterns and 37 in the female foot patterns. Additional pressure rods 3, 8, and 10 are provided on the board as well as holes for accomodating still more such rods. Special pressure rods for intense massaging are utilized at the reflex points for the genital gland, abdominal neuropathy, lymphatic gland, the stomach, intestines and duodenum.

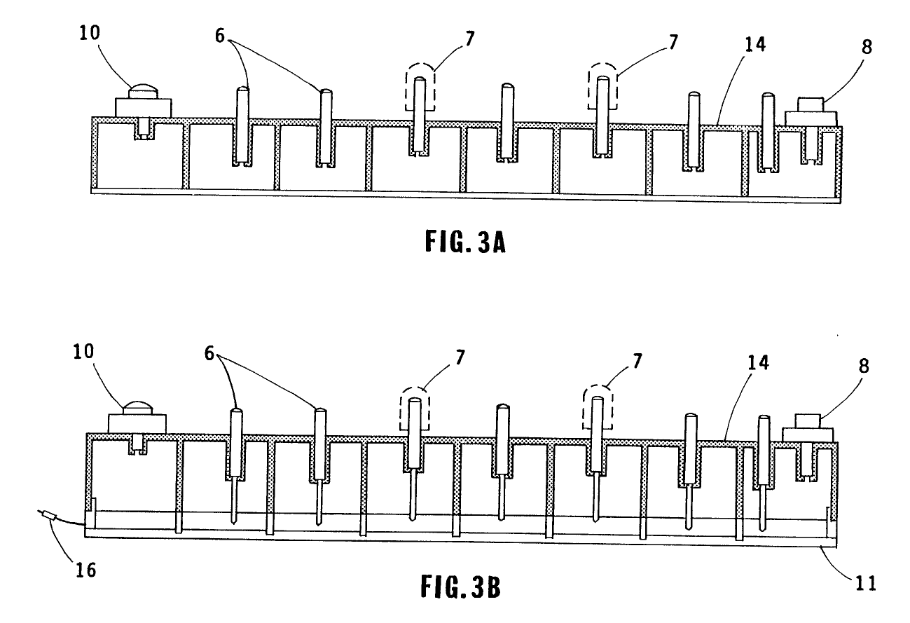

Pressure caps 7 can be taken from the stand 4 and installed on the tops of normal pressure rods to increase the height of rods, as may be called for. Also, an adapter 16 as shown in FIG. 3B can be utilized for thermal and vibrating pressure rods. The electrical circuitry for the adapter is installed on the underside 11 of the board.

The normal pressure rods 6 can be replaced with thermal pressure rods 21 and vibrating pressure rods 22, as shown in FIG. 2A. These rods are electrically connected to control circuitry (not shown), powered through power plug 16 and power supplied through switch 13. Switch 12 is used to switch between thermal and vibration operation. Thermal and vibration generators for acupressure systems are well known in the art.

Rod 18 (FIG. 2A) is a rounded relatively painless rod which may be utilized by a new user adapting to the use of the device. Rods 19, 20 and 23 are jade, ocher, and magnetic pressure rods, respectively which may be installed instead of the normal rods should the user so prefer.

The device of the invention thus enables acupressure treatment to reflex points on a users foot which places pressure rods in accordance with average foot sizes so that they massage the correct reflex points. The pressure provided can be varied for optimum results by selecting pressure rods of different heights and thicknesses. Further partial areas can be intensely massaged with sets of rods placed independent of the foot patterns.

While the invention has been described and illustrated in detail, it is to be understood that this is intended by way of illustration and example only, the scope of the invention being limited by the terms of the following claims.

I claim:

1- An acupressure foot board for massaging the soles of a human foot at the reflex points on said sole comprising:
a base board, the outline of at least one foot sole having dimensions approximately that of a human foot sole formed on the surface of said board;
a plurality of holes formed in said board within the foot outline, each of said holes being located at a spot on said foot sole outline corresponding to a reflex point on the sole of a human foot; and
a pressure rod installed in each of said holes, said pressure rods operating to massage the reflex points on said sole.

2- The acupressure foot board of claim 1 wherein there are two pairs of said foot outlines, one of said pairs being of a larger size for use by men and the other pair being of a smaller size for use of men and the other pair of a smaller size for use by women and children.

3- The acupressure foot board of claim 1 and further including pressure caps placed on preselected ones of said pressure rods to extend the length and increase the massaging pressure thereof.

4- The acupressure foot board of claim 1 wherein the tops of said pressure rods are rounded to place minimum pressure on the soles of the user.

5- The acupressure foot board of claim 1 wherein said rods are temperature rods which are heated.

6- The acupressure foot board of claim 1 wherein said rods are vibrator rods which are vibrated in performing massaging of foot soles.

7- The acupressure foot board for massaging the soles of feet at the reflex points thereon comprising:
a base board;
the outlines of two pairs of foot soles being formed on the surface of said board, one of said pairs of said sole outlines being larger in size and corresponding to the soles of an average man, the other of said pair of said outlines being smaller in size and corresponding to the soles of an average women or child;
a plurality of holes being formed in said board within each of said foot outlines, each of said holes being located at spot on the foot sole outline corresponding to a reflex point on the sole of a human foot;
a pressure rod installed in each of said apertures for massaging the reflex points on the user's foot sole; and
a plurality of caps which may be fitted onto selected ones of said pressure rods to increase the lengths thereof and the degree of massaging.

8- The acupressure foot board of claim 7 wherein said rods are thermal rods which add heat to the massaging action.

9- The acupressure foot board of claim 7 wherein said rods are vibrating rods which add vibration to the massaging action.

10- The acupressure foot board of claim 7 wherein the outlines of said foot soles are formed on foot shaped film attached to the board.
